(19) United States
(12) Patent Application Publication
(10) Pub. No.: US 2008/0058779 A1
(43) Pub. Date: Mar. 6, 2008

(54) Method for Affecting Biomechanical Properties of Biological Tissue

(75) Inventors: AnnMarie Hipsley, Silver Lake, OH (US); Lucinda Camras, Omaha, NE (US)

Correspondence Address: Daniel J. Schule, Suite 500, 388 S. Main Street, Akron, OH, 44311, US

(73) Assignee: Ace Vision USA, Silver lake, OH (US)

(21) Appl. No.: 11/850,407

(22) Filed: Sep. 5, 2007

Related U.S. Application Data
(60) Provisional application No. 60/842,270, filed on Sep. 5, 2006.

(51) Int. Cl. A61F 9/008; A61B 18/20

(52) U.S. Cl. 606/4; 606/14; 607/89

(57) ABSTRACT

A method for changing at least one biological property of a biological tissue, the method having the steps of providing a biological tissue having at least a first layer; evaluating the topography, biomechanical and fundamental properties of the biological tissue by layer; and orienting a matrix complex in the biological tissue, wherein the matrix complex has matrices that are balanced in a mathematical relationship, wherein each of the matrices comprises a perforation formation in the tissue, and wherein the mathematical relationship comprises a mathematical algorithm.

### RELATED APPLICATIONS

This application claims priority to U.S. Patent Application Ser. No. 60/842,270, entitled METHOD FOR AFFECTING BIOMECHANICAL PROPERTIES OF BIOLOGICAL TISSUE, filed Sep. 5, 2006. Furthermore, this application hereby incorporates by reference all of the subject matter of U.S. Patent Applicant's Ser. No. 60/842,270.

### BACKGROUND

Corrective eye procedures are well-known, and the art has now advanced to the stage at which self-contained laser based systems are sold as stand alone units to be installed in a surgeon's operatory or a hospital, as desired. Thus, hospitalization is not necessarily required in order to perform such ophthalmological surgery.

Such systems typically include a p.c. (personal computer) type work station, having the usual elements (i.e., keyboard, video display terminal and microprocessor based computer with floppy and hard disk drives and internal memory), and a dedicated microprocessor based computer which interfaces with the p.c. work station and appropriate optical power sensors, motor drivers and control elements of the ultraviolet laser, whose output is delivered through an optical system to the eye of the patient.

In use, after the patient has been accommodated on a surgery table or chair, the system is controlled by the operator (either the surgeon or the surgeon and an assistant) in order to prepare the system for the delivery of the radiation to the patient's eye at the appropriate power level and spatial location on the corneal surface.

In some systems, a provision is made for permanently recording on a plastic card made of PMMA (polymethylmethacrylate) a spot image of the laser beam used in the surgical operation. This spot is recorded prior to the operation to ensure that the beam power is properly adjusted and to provide a permanent record of the beam used. PMMA is typically used due to the characteristic of this material of having a closely similar ablative photodecomposition response to that of the human corneal tissue. After the surgery has been performed, the resultant data is typically made part of a permanent record, which becomes part of the patient's file.

The utility of such ophthalmological surgery is well-known, and there is therefore a need for new methods directed to improving ophthalmological surgery even further.

### BRIEF DESCRIPTION OF THE DRAWINGS

In the accompanying drawings, which are incorporated in and constitute a part of the specification, embodiments of the invention are illustrated, winch, together with a general description of the invention given above, and the detailed description given below, serve to exemplify embodiments of the invention:

FIG. 1 is a cross-sectional view of the human eye and the optics relating to it.

FIG. 2 is a cross-sectional view of the human eye and the optics relating to it.

FIG. 3 is a cross-sectional view of the human eye and general biomechanics during accommodation.

FIG. 4 is in exploded view of the human eye and the biomechanical strain on sclera.

FIG. 5 is a cross-sectional view of the human eye and general biomechanics during accommodation.

FIG. 6a is a cross-sectional view of biological tissue having perforations therein.

FIG. 6b is a perspective view of biological tissue having nine perforations therein.

FIG. 7 is a perspective view of the energy storage, shock absorption, and elasticity restoration of perforated biological tissue.

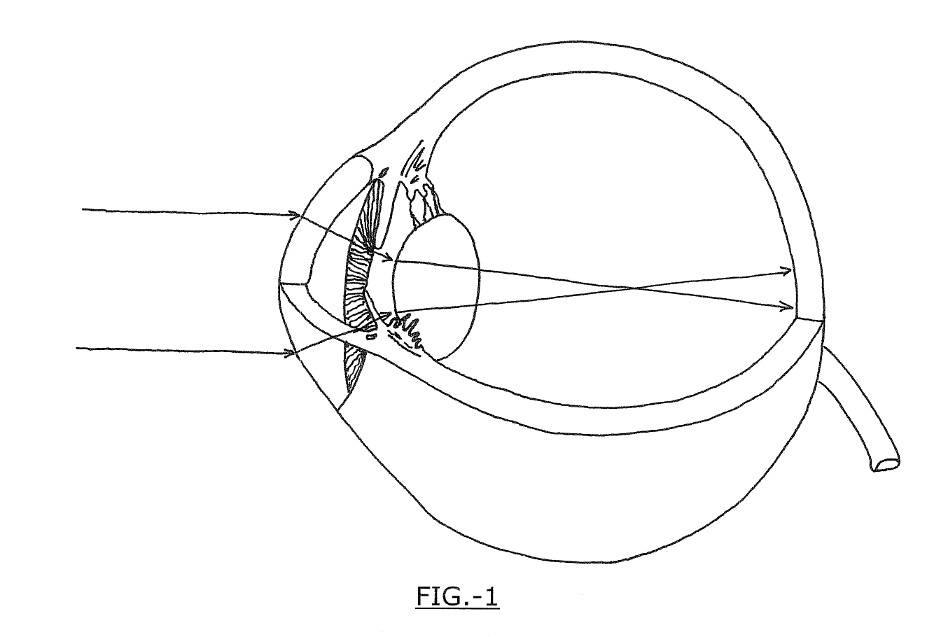

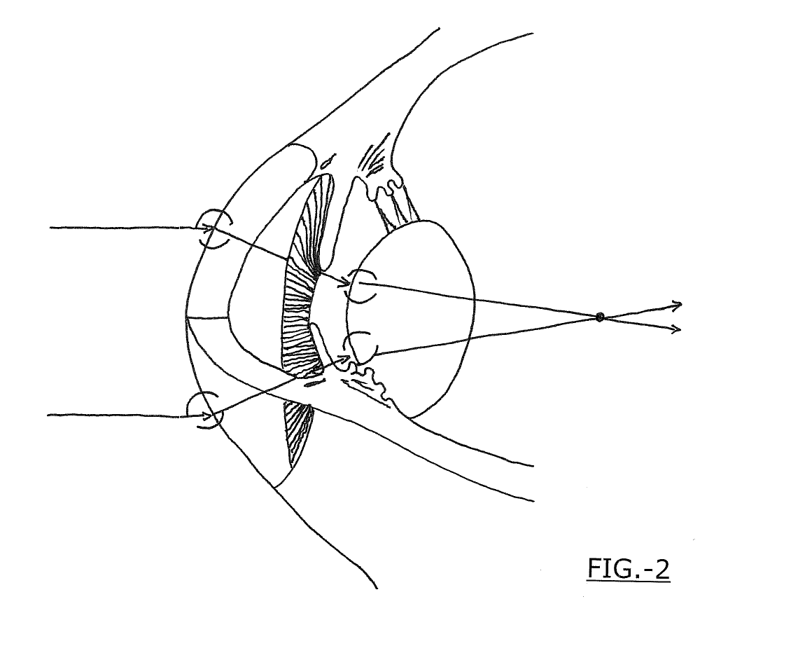

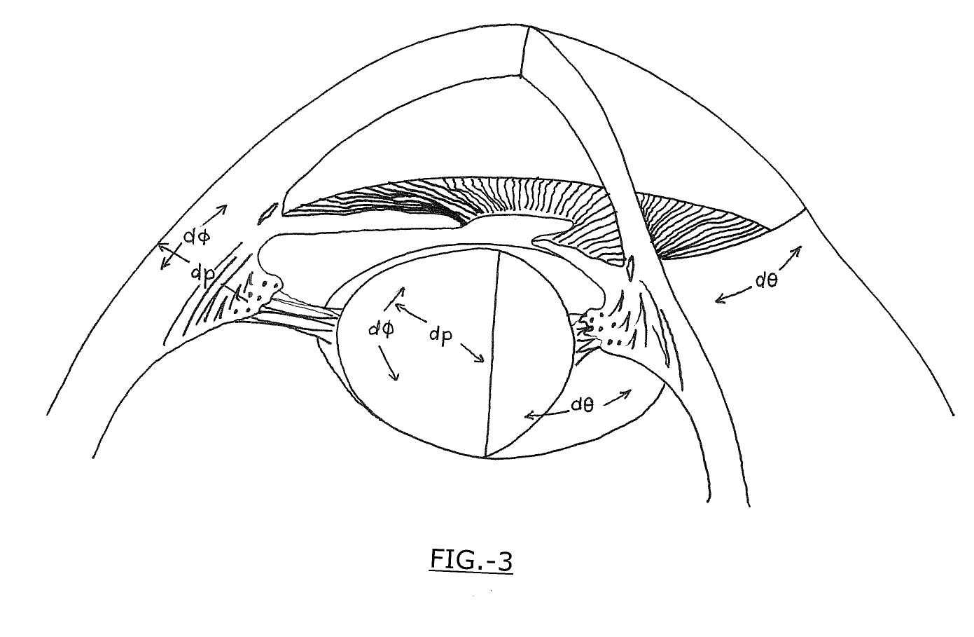

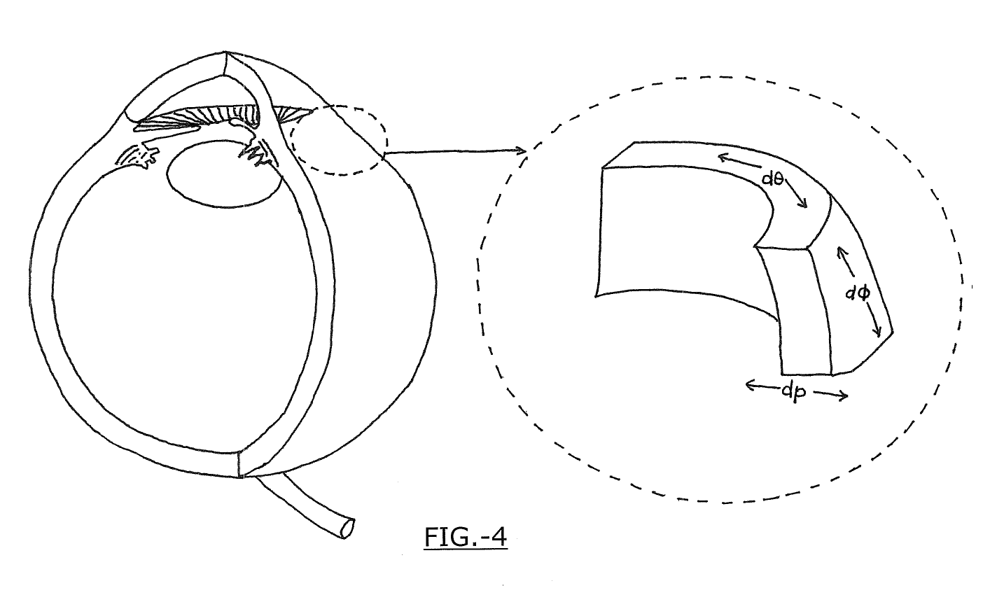

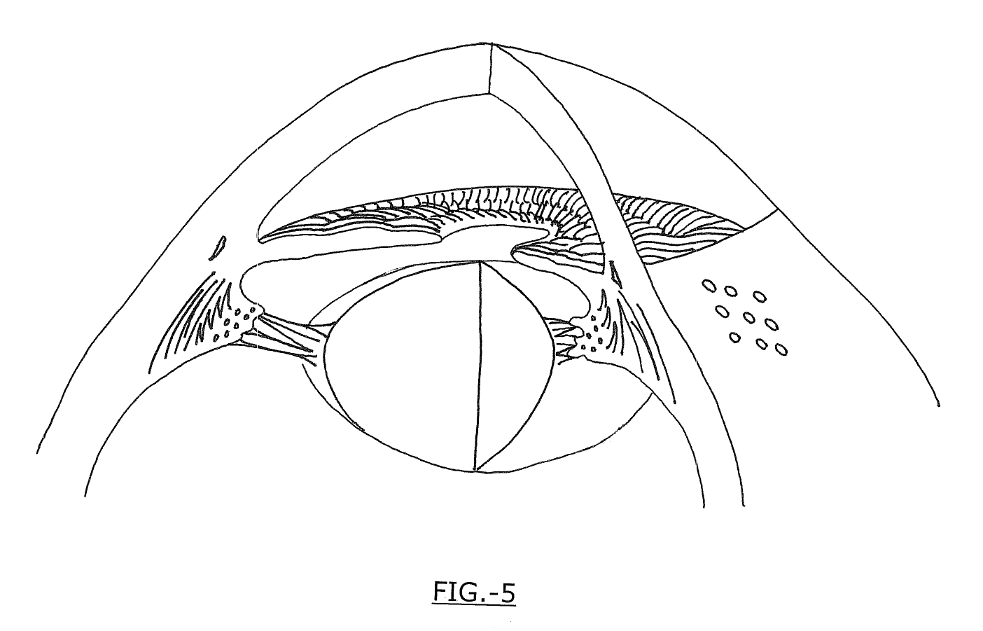

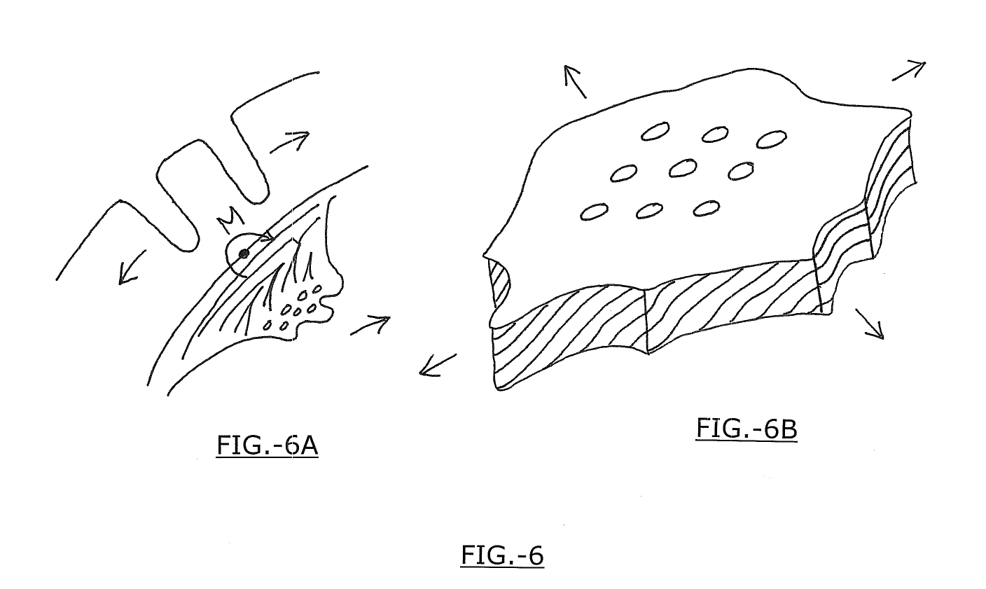

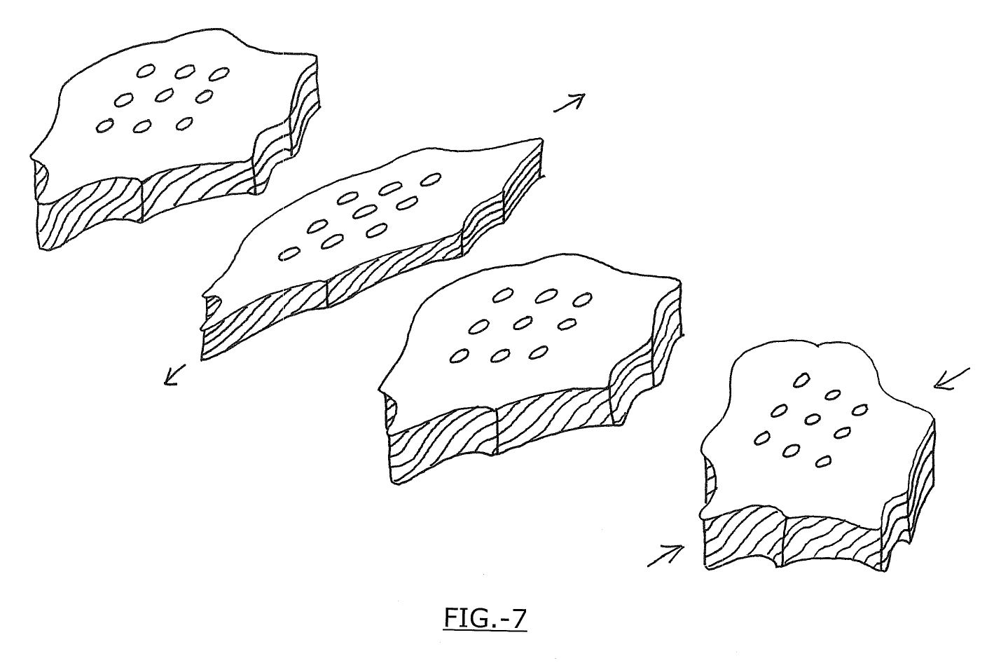

### DETAILED DESCRIPTION

Embodiments are directed to a method for changing a least one biological property of a biological tissue, by means of orienting in the biological tissue a matrix complex (which may be referred to as a biomatrix), which is a complex of at least one, matrix arranged in an orientation with respect to other matrices in the complex. The orientation of the matrices in the matrix complex is governed by a mathematical relationship that comprises a mathematical algorithm. It is desirable to achieve a change in at least one biological property without substantially undermining the structural integrity of the biological tissue. Indeed, it is within the scope of the present invention to actually improve the overall structural integrity and/or operational functionality of the biological tissue by means of introducing the matrix complex (described below) into the biological tissue. Exemplary of the present invention, the method may be practiced for the purpose of achieving decompression or soft tissue release of load bearing or constricted (or contracted) connective tissues.

For purposes of this invention, a matrix comprises a plurality of perforations (at least two, and preferably three or more) of the biological tissue, wherein the perforations are arranged in a pattern or mathematical orientation with respect to each other, to form the matrix. The perforations form lattice points or partitions within the biological tissue in the area of the matrix.

A perforation is the result of the selective removal or manipulation of tissue, by excision, incising, vaporization, and ablation. The perforation will preferably extend from the surface of the biological tissue into the biological tissue. The perforation(s) may extend entirely through the biological tissue or only partially through the biological tissue. The perforation may extend through one or more sub-tissues (described below). The perforation may have the form of a pore, having a first end adjacent the surface of the tissue and second end, which may be at a depth x into the tissue wall, and a pore wall extending from the first end to the second end. The perforation may be substantially cylindrical; however, any shape may be selected with sound judgment. The formation of a perforation will result in the removal of a volume of tissue, which volume may be calculated with respect to the physical dimensions of the pore itself, including cross sectional area and depth. According to one aspect of the invention, the volume of tissue removed in each perforation and/or the total volume of tissue removed in the sum of all perforations in the matrix complex may bear a proportional mathematical relationship to the biological property being affected, such that it will be possible to limit or control the change in the biological property by limiting or controlling the volume of tissue removed in the matrix complex.

The perforation may be formed by cutting, incinerating, or vaporizing the tissue by mechanical, light, chemical or atomic force means. Other suitable means of forming the perforation may also be selected. In a preferred embodiment, the perforation may be formed by means of a laser. Laser vaporization of target soft tissue has very little effect on surrounding soft tissue structures or tissues.

Laser features may include:
i. Er:Yag specifications in the 2.94 um range
ii. any laser in the full range of the light spectrum
iii. nano or biofilms applications to create porous surfaces
iv. chemical or biochemical applications
v. fiberoptic system OR colluminated arm system to deliver 2.94 um wavelength
vi. custom tips
vii. Free electron Laser or FEL Er:Yag system
viii. Handpiece can be attached or remote
ix. Handpiece may or may not include a probe. (Shen) with or without a feedback mechanism, with or without laser controls incorporated into the handpiece
x. Nanotechnology application for scanning, feedback & delivery of predetermined specifications of tissue removal
xi. 3D images & nano images assisted visualization of soft tissue may or may not be used
xii. Tips can be quartz, sapphire, or other in contact or non contact

The method of the present invention comprises the step of providing a biological tissue. Suitable biological tissues may include muscle tissue, connective tissue, fascia, scar tissue, skill, cartilage and bone. The present disclosure does not intend to limit the type of biological tissue for which the methods may be applied. Though the invention contemplates the use of biological tissue, it is recognized that the methods may be practiced in whole or in part on non-biological tissues for the purposes of achieving an effect on the physical and structural properties of the non-biological tissues. In the preferred embodiment, the biological tissue is the sclera or other soft or connective tissues of the eye.

It will be understood by one of ordinary skill in the art that a biological tissue will have certain physical/mechanical and biological properties, which influence the operation of the biological tissue internally (i.e., within itself) and within its immediate environment (i.e., in relation to other surrounding structures and tissues). These properties (collectively referred to as “biomechanical properties”) may include elasticity, shock absorption, resilience, mechanical dampening, energy storage, pliability, stiffness, rigidity, configuration, alignment, deformation, mobility, and volume. In addition to these biomechanical properties, biological tissues may have a complex structure, which may include a plurality (two or more) of layers of different types of sub-“tissues”, which work in conjunction with each other to moderate the function of the biological tissue. Though biological tissue may have a complex structure, it is generally considered a continuous medium.

The biomechanical properties of biological tissue may change over time or in response to a variety of factors, including injury, illness, repetitive movement (fatigue), and age. For example, biological tissues may lose or gain flexibility or rigidity over time, resulting in all adverse impact on tissue function. According to embodiments of the present invention, biomechanical properties of biological tissues may be altered by a plurality of excisions (perforations) of biological tissue to remove a total pre-selected volume of excised tissue. It is believed that the location of the perforations in the biological tissue and the volume of tissue removed in each perforation, and the sum of all perforations, is particularly relevant to the impact such excisions have on the integrity of the biological tissue (i.e., maintaining structural integrity and function, amongst others) and the amount of change in the biomechanical properties of the tissue. For example, in one embodiment, the volume of the individual pores or perforations removed may be such that the overall change in density is proportional to the decrease in load stress, where load stress is the biomechanical property being affected.

It is believed that the affect of perforating the biological tissue influences biomechanical properties, such as structural pliability, by creating areas of flexibility wherein the perforations act as flexible “diaphragm pumps” which significantly change the extensibility of tissues in critical anatomical areas of significance thereby restoring biomechanical efficiencies of the physiological structures/tissues/systems being released from the impingement.

Stated another way, the excision of tissue according to the present disclosure produces areas within the biological tissue where previously homogenous tissue is altered to tissue having areas of positive and negative stiffness. As noted above, biological tissue has distinct properties and structural hierarchy in its design, architecture and function. In addition, biological tissue may occur in vivo in layers and planes, akin to an onion. The matrix complex of the present invention changes the architecture of the biological tissue by changing what was a continuous medium into a heterogeneous tissue with areas of perforation. The result is a change in mechanical structure, behavior, and function.

As indicated above, the present invention discloses an arrangement, and a method of determining the arrangement, of perforations to form one (and preferably two or more) matrices and the arrangement, and method of determining the arrangement, of matrices within a matrix complex oriented on the biological tissue, to achieve a change in at least one biomechanical property of the biological tissue. Moreover, the present invention discloses a method for selecting the number and size of perforations to achieve a desired change in at least one biomechanical property of the biological tissue.

Having provided a biological tissue, the method may include evaluating the topography, biomechanical and fundamental properties of the biological tissue layer by layer, as appropriate. As discussed above, a biological tissue may have more than one layer, like an onion. The term “fundamental properties” refers to original structure, nature and composition. Evaluation of the topography (i.e., the physical structure of the tissue layers, including depth, surface area, and the like) and biomechanical and fundamental properties of each layer of the biological tissue provides data concerning the overall structure of the biological tissue and is relevant to determining the disparate effect on the sub-tissues that a perforation extending through multiple sub-tissues may impart. Evaluation of the topography of the biological tissue and its sub-tissues may be achieved by as described herein. Evaluation of the biomechanical properties of the biological tissue and its sub-tissues may be achieved by as described herein. It will be understood that evaluating and characterizing the biomechanical properties of the biological tissue will include determining the physical constants and forces operating in the tissue, including elastic constants and atomic forces, which may vary between biological tissues and between sub-tissues within a biological tissue.

The method further comprises orienting a matrix complex in the biological tissue, where the matrix complex is comprised of one, and preferably, more than one matrix of perforations. The matrix complex may be oriented with respect to the biological tissue so that the constituent matrices are substantially “balanced” within the tissue. By “balanced” it is meant that the matrices are position in such an orientation that the region-specific effects on biomechanical properties in the areas in and adjacent each matrix are balanced across the entire biological tissue so as to achieve the desired change in the biomechanical property of the biological tissue as a whole, without adversely affecting the integrity, or other desirable structural characteristics of the tissue. It is particularly desirable that the balance results in a substantial equilibrium of forces in the biological tissue under both static and dynamic conditions. It is further desirable that the matrix complex is positioned with respect to the biological tissue to reduce shearing effects between the individual matrices in the matrix complex and between the perforations within the matrix. This balancing may result in an increase to the integrity of the biological tissue.

To this end, the matrices forming the matrix complex may be positioned according to a mathematical relationship comprising a mathematical algorithm. This mathematical relationship and the associated algorithm will be described in further detail below. It will be understood that the matrix complex may, in an alternate embodiment, be unbalanced, though a balanced arrangement is preferred.

Now the mathematical relationship of the matrices within the matrix complex will be described. In one embodiment, the mathematical relationship governing the positioning of the matrices within the matrix complex is based on a mathematical algorithm. The mathematical algorithm may use a factor of Φ (Phi) to create a model of the biological tissue to find the most efficient placement of the matrices to alter the biological property of the biological tissue. For purposes of this application, the factor Phi is 1.618 and represents any fraction of a set of spanning vectors in a lattice having the shortest length relative to all other vectors' length. The factor Phi is believed to be particularly relevant to the organizational structure and development of many biological tissues, organs, and larger bodies, being a ratio that tends to appear repeatedly in nature. In one embodiment, the algorithm may use a factor of Phi, however, the algorithm may use a derivative of Phi, such as, but not limited to, an addition of, a subtraction of, a multiplication of, a division of, all exponent of, a root of, or such other mathematical derivative of, Phi.

The mathematical algorithm may additionally or alternatively include an atomic relationship factor, which may comprise a predictable relationship between the volume removed in each perforation and the change in the fundamental and biomechanical property of the biological tissue. This is described briefly above. In one embodiment, the relationship of the removed volume to the magnitude of the change in the biomechanical property of the tissue may be mutually exclusive.

The mathematical algorithm may include a non linear hyperbolic relationship between planes of biological tissue and at any boundary or partition of neighboring tissues, planes and spaces in and outside of the matrices. However, the relationship may be linear, nonlinear or Euclidean or non-Euclid space.

As previously discussed, each matrix comprises an array of perforations in a pattern. The matrix may properly be depicted mathematically as a point lattice, in which the perforations comprise points in the lattice. It will be understood that each matrix will comprise rows and columns of perforations, and that each row will have a length and each column will have a length. Within each matrix and between the perforations in a matrix, there may be one or more mathematical relationships which govern the positioning of the perforations with respect to each other, and within the matrix. For example, according to one embodiment, the row length and column length of the matrix may correlate to each other by means of a linear algebraic relationship. The linear algebraic relationship may be described as a vector relationship following planar and Euclidian geometry. According to another embodiment, each perforation in the matrix may have continuous linear vector spaces with derivatives up to infinite number relationship “n”. Still further, given that each matrix has a surface area calculable with reference to its row lengths and column lengths and given that the matrix complex covers a total surface area representing the sum of the matrix surface areas, each perforation may have a proportional relationship to the matrix complex surface area. Each perforation may further bear a linear relationship with one, more than one, or all of the other perforations in the matrix.

It will be understood that each matrix may be defined either or both two-dimensionally and three-dimensionally. The two-dimensional surface appearance of the matrix will be defined by the openings of the plurality of perforations in the matrix. In one embodiment, these openings may be arranged in a pattern such that the matrix is tessellated. The tessellation may comprise a repeating, or a non-repeating pattern, or a blend of both repeating and non-repeating patterns. The tessellations may be one or a combination of more than one of Euclidian, non-Euclidian, regular, semi-regular, hyperbolic, parabolic, spherical, or elliptical tessellations. Mathematics relating to each of these tessellations is known. In one embodiment, the tessellation may be a square having the property of being able to be subdivided into a tessellation of equiangular polygons to a derivative of n. Due to the linear relationship of the matrices, the constant of phi can be used in biological tissue as a tissue architectural ruler in creating a virtual 3D archetype which can correlate to a mathematical algorithmic code which can be translated by computer via a software program. According to another, related embodiment, the tessellation may comprise an infinite number of tessellating tetrahedrons within the square for desired tissue effect and volumetric requirement. In other words, given the desired volume of tissue that needs to be removed to achieve a given change in the biological tissue, the number of individual perforations, which define the boundaries of the tessellation, will be restricted largely by the technological limitations in removing a volume of tissue at the perforation. It will be understood that present technology limits the feasibility of such an “infinite” tessellation, but that the number of tessellations may be finite.

The tessellation may be directly or indirectly related to stress or shear strain atomic relationships within the biological tissue. In addition or in the alternative, the tessellation may be directly or indirectly related to stress or shear strain atomic relationships between the biological tissue and its surrounding tissues. The relationship may be defined with respect to a mathematical array of position vectors between perforations. According to another aspect of the invention, the method of the present invention may include computing the mathematical array of position vectors between perforations.

In light of the large number of mathematical calculations governing the location of the matrix complex, the matrices, and the perforations in the matrices, the methods of the present invention further contemplate the provision a software program adapted to calculate the location of the matrix complex on the biological tissue and the location of the perforations within the matrices, all using the mathematical algorithm and related to (accounting for) the structural hierarchy of the biological tissue, which may be determined by understanding the homogeneity of layers of the biological tissue. The software program will preferably be adapted to calculate the location of the perforations on the tissue according to the algorithm and variables described above, including the respective pore depth and size, to achieve orientation of the matrix complex on the biological tissue having sufficient volume excision to achieve the desired change in the biomechanical property of the tissue.

In another embodiment, the software program may transmit the location of the matrix complex and the perforations to a tissue perforating device. And in still a further embodiment of the invention, the software program may additionally transmit control instructions to the tissue perforating device. The control instructions refer to the instructions that guide the tissue perforating device (laser) around the biological tissue and govern the operation of the tissue perforating device.

In one specific embodiment of the method involving treatment of the sclera, a predetermined pattern of perforations in critical zones in the scleral tissue of the eye is believed to proportionally decrease load stress/shear stress of the scleral tissue of the eye globe which has become contracted due to aging or some other process which interferes or disrupts the normal biomechanical and physiological properties and processes of the functions of the eye. The “micro expansion” in the critical areas is small enough that it does not significantly change the overall diameter or expansion of the entire globe of the eye. The mathematical model for scleral decompression is based on age related geometric and tissue characteristics. The scleral decompression is accomplished by removing strategic areas of sclera in opposing quadrants of the anterior globe segment whereby a series of connective tissue partitions are created using a non contact method and Er:YAG 2.94 um wavelength. The resulting “gap junctions” can be created in a number of design patterns, however, they will preferably have a critical width which is necessary to prevent scleral residual tissue approximation thereby inhibiting normal fibroblast activity retarding the connective tissue healing cycle. Collagen reorganization is loosely organized and less differentiated. It is believed that this strategic removal of scleral tissue creates “flexible diaphragm pumps” that act as extensible dividing membrane partitions allowing for decompression of scleral load stress and restoration of biomechanical efficiency in the tissues that interact in the internal and dynamic functions of the eye including but not limited to: accommodative system (lens function), hydrodynamic system (aqueous outflow), neuromuscular system (afferent/efferent visual pathways), neurovascular system (choroidal/retina functions). It is believed that the volume and location of tissue ablation is relevant to achieving areas of decreased load stress, decreased tissue strain and deformation, as well as decompression of receptors necessary for maintaining the homeostasis of the fluidic, dynamic, and chemical processes both extracellular and intracellular. It is also thought that the effects of the Er:Yag Laser wavelength and mechanism of action produces slight desirable thermal and mechanical effects which denature the collagen tissue fibers of the residual sclera and retard or even inhibit the healing process. The newly created “flexible diaphragm pumps” restore the normal biomechanical efficiency of the underlying dynamic systems and allow a return of function.

This method of treatment in this embodiment may entail vaporizing a predetermined volume of scleral tissue either in the anterior or posterior eye globe in critical zones. This method is the vaporization of scleral tissue as calculated proportionally by segmental zones of micro-cylinders or micro-prisms with specific emphasis on decompression of scleral load stress in three critical zones in the anterior globe. The described distances are radial measurements from the visual axis: proximal zone is ≧0.5 mm from the posterior surgical limbus and ≦0.8 mm central zone is equidistant between proximal zone and distal zone distal zone is just anterior to the annulus of inn or the insertion of the EOM specifically: at least 5.0-5.5 mm from the posterior surgical limbus.

In this embodiment (specific to removing sclera from the anterior globe), the approximation of the inner wall edge of the individual pore or perforation produced may be no less than 400 um and ideally is greater than or equal to 600 um. This is to disrupt the normal tissue healing response and to retard fibroblast formation disallowing cross-bridging and thus maintaining the maximum amount of evacuated tissue despite eventual tissue healing. In another embodiment tissue adjacent the posterior globe may be altered according to the methods of the present invention.

The minimum depth in scleral tissue should be calculated to produce full thickness micropuncture to the subchoroidal lamina to improve uveal-scleral aqueous flow to have an effective decrease effect in IOP. If soft tissue layers are released microscopically through the subchoroidal level, all immediate decrease in average of 3-mmHg IOP should be achieved. This depth for the average scleral thickness is approximately equal to or greater than 600 um. This should be calculated as f(x) whereas x=scleral depth. Scleral depth is regionally specific in the globe. The total spherical volume removed per quadrant is calculated in relationship to the tangential axis of the globe and as a function of scleral depth in the specific globe zone location. Proportional volume will be removed in perforations whereas the overall surface area should not exceed the width of 5.0 mm and the length of 5.0-5.5 mm and depth. The shape of the overall surface representation of the “dot matrix” of perforations is variable and may exist in a variety of patterns, including a “cross Pattern” or diamond pattern. This diamond pattern can be further described as a series of tessellated equilateral triangles or 3D tetrahedral series.

The biological tissue may have a range of isotropic elastic constants across the medium. In such an embodiment, the matrices have a position within the matrix complex, wherein the position of the matrices is selected to create a non-monotonic force deformation relationship in the biological tissue.

The embodiments have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

What is claimed is:

1. A method for changing at least one biological property of a biological tissue, the method comprising the steps of:
providing a biological tissue having at least a first layer;
evaluating the topography, biomechanical and fundamental properties of the biological tissue by layer; and
orienting a matrix complex in the biological tissue, wherein the matrix complex has matrices that are balanced in a mathematical relationship, wherein each of the matrices comprises a perforation formation in the tissue, and wherein the mathematical relationship comprises a mathematical algorithm.

2. The method of claim 1, wherein the at least one biological property is elasticity, shock absorption, resilience, mechanical dampening, pliability, stiffness, rigidity, configuration, alignment, deformation, mobility, or tissue volume.

3. The method of claim 1, wherein the biological tissue has a range of isotropic elastic constants across the medium; and
wherein the matrices have a position within the matrix complex, wherein the position of the matrices is selected to create a non-monotonic force deformation relationship in the biological tissue.

4. The method of claim 1, wherein each matrix has a row length and a column length,
wherein there is a linear algebraic relationship between the row length and the column length; and
wherein each perforation in the matrix formation has continuous linear vector spaces with derivatives up to infinite number relationship “n”.

5. The method of claim 4, wherein each matrix has a surface area and wherein the sum of the surface areas of the matrices is the matrix complex surface area; and
wherein each perforation has a proportional relationship within the matrix and matrix complex surface area.

6. The method of claim 1, wherein the matrix complex is positioned with respect to the biological tissue to achieve within the biological tissue a substantial equilibrium of forces in both static and dynamic conditions.

7. The method of claim 6, wherein the matrix complex is positioned with respect to the biological tissue to reduce shearing effect between the matrices within the matrix complex and between the perforations within the matrix to increase the integrity of the tissue.

8. The method of claim 1, wherein each perforation has a linear relationship with the other perforations within each matrix and the complex of matrices individually.

9. The method of claim 1, wherein the perforation is formed by excising a volume of the biological tissue.

10. The method of claim 9, wherein the shape of the excised volume is substantially cylindrical.

11. The method of claim 9, wherein each perforation defines a point within each matrix and matrix complex on the surface of the biological tissue.

12. The method of claim 1, wherein the matrices are tessellated.

13. The method of claim 12, wherein the tessellation comprises a repeating pattern and tessellations are Euclidian, non-Euclidean, regular, semi-regular, hyperbolic, parabolic, spherical, or elliptical and any variation therein.

14. The method of claim 13, wherein the tessellation comprises a non-repeating pattern and tessellations are Euclidian, non-Euclidean, regular, semi-regular, hyperbolic, parabolic, spherical, or elliptical and any variation therein.

15. The method of claim 13, wherein the tessellation is directly related to one or more of stress or shear strain atomic relationships within the biological tissue and between the biological tissue and its surrounding tissues by a mathematical array of position vectors between perforations.

16. The method of claim 15, further comprising the step of computing the mathematical array of position vectors between perforations.

17. The method of claim 13, wherein the tessellation is indirectly related to one or more of stress and shear strain atomic relationships between tissues.

18. The method of claim 15, wherein the mathematical algorithm comprises an atomic relationship factor, wherein the atomic relationship factor comprises a predictable relationship between the volume removed in each perforation and the change in the biological property of the biological tissue.

19. The method of claim 18, wherein the relationship of the removed volume to the magnitude of biomechanical change in the tissue is mutually exclusive.

20. The method of claim 12, wherein the tessellation is a square having the property of being able to be subdivided into a tessellation of equiangular polygons to a derivative of n.

21. The method of claim 20, wherein the tessellation comprises all infinite number of tessellating tetrahedrons within the square for desired tissue effect and volumetric requirement.

22. The method of claim 20, wherein the tessellation comprises a finite number of tessellating tetrahedrons within the square for desired tissue effect and volumetric requirement.

23. The method of claim 1, wherein the mathematical algorithm uses a factor of Φ or Phi to find the most efficient biological mapping for the proportionate placement of the matrices to alter the biological property of said biological tissue.

24. The method of claim 23, wherein the factor is selected from the group consisting of an addition of; a subtraction of, a multiplication of, a division of, an exponent of, and a root of, Phi

25. The method of claim 23, wherein the factor of Φ or Phi is 1.62 and represents any fraction of a set of spanning vectors in a lattice having the shortest length relative to all other vectors' length.

26. The method of claim 23, wherein the mathematical algorithm includes a non linear hyperbolic relationship between planes of biological tissue and at any boundary or partition of neighboring tissues, planes and spaces in and outside of the matrices.

27. The method of claim 23, further comprising the step of providing a software program adapted to calculate the location of the matrix complex on the biological tissue and the location of the perforations within the matrices, all using the mathematical algorithm, related to the structural hierarchy of the biological tissue.

28. The method of claim 27, wherein the structural hierarchy is determined by understanding homogeneity of layers of biological tissue.

29. The method of claim 27, wherein the software program transmits the location of the matrix complex and the perforations to a tissue perforating device.

30. The method of claim 29, wherein the software program further transmits control instructions to the tissue perforating device.

31. The method of claim 29 wherein the tissue perforating device has laser or scanning device that contains a biofeedback mechanism either within the head of the laser or within the device.

32. The method of claim 30, wherein the tissue perforating device is a laser.

33. The method of claim 1, wherein the biological tissue is sclera.

34. The method of claim 1 wherein the biological tissue is any arterial lumen or nervous tissue lumen or sheath.
